(19) United States
(12) Patent Application Publication
(10) Pub. No.: US 2002/0013598 A1
(43) Pub. Date: Jan. 31, 2002

(54) Body piercing decoration system

(76) Inventor: Raimond Lasar, Rambach (DE)

Correspondence Address: LAWRENCE ROBERT BRANDT, P.O. BOX 1937, SAXONVILLE, MA, 01701, US

(21) Appl. No.: 09/803,563

(22) Filed: Mar. 9, 2001

(30) Foreign Application Priority Data
Mar. 9, 2000 (DE) G10011585.3
Dec. 11, 2000 (DE) G20018811.9

(51) Int. Cl. A61B 17/34

(52) U.S. Cl. 606/188

(57) ABSTRACT

A body decoration system (26) for the application of decorative sections (1) by piercing the body on artificial openings of the body, preferably, including a body-piercing section (19) that is inserted into the artificial body opening so that one end of the body-piercing section (19) protrudes out on either side of the body opening. In addition, the body decoration system includes a fastening section (24) that has a larger diameter than the body-piercing section (19) such that when these two parts are joined to each other it is not possible for the body-piercing section (19) to be pulled out or slip out in one direction. In addition the body decoration system includes a head section (9) for fastening a decorative section (1). The head section (9) also attaches to the body-piercing section (19). The decorative section (1) can be fastened securely to the head section (9), but can also be removed so that it can be exchanged with another decorative section (1) without having to remove the body-piercing section (19) from the artificial body opening, and without having to detach the head section (9) or the fastening section (24) from the body-piercing section (19).

### CROSS-REFERENCE TO RELATED APPLICATIONS

None

### BACKGROUND

1. Field of the Invention

The invention concerns a system for producing body decorations in general and a system for utilizing body piercings for decorative purposes in particular.

### BACKGROUND

2. Description of Prior Art

People have had the desire to adorn their bodies since time immemorial, and so today there exists a wide range of decorative elements and methods for body decoration. Many of these are applied immediately to the human body, for example rings, earrings, as well as body piercing pins, which have become increasingly popular in recent years, and which are used to pierce various parts of the body.

For body piercing, an opening or channel is first pierced through the human tissue to allow a body-piercing pin to be fastened in place, similar to how an earring pin is attached. A typical body-piercing pin used for this purpose will have a curved rod that is pierced through the artificially created opening in the body. At one end of the rod is a decorative element, for example a gem or silver, gold, platinum or niobium jewelry. After the rod has been pushed through the body opening, a securing ball is screwed onto the opposite end of the rod so that the body-piercing pin cannot slip out of the body opening.

One of the disadvantages of the body piercing systems and devices that are currently in use is that the entire body-piercing pin must be removed in order to replace the body decoration. This is because the decorative element is most often fixed to an end of the body piercing section. In order to replace a body decoration, the securing ball is screwed off the body piercing section and the rod is pulled back out of the opening in the body to then insert another body piercing pin in place as was described above. This procedure is awkward for parts of the body that are accessible only with difficulty and may involve pain and the risk of inflammation for the person with the piercing. Compared to standard earrings, pain and risk of inflammation may be greater with larger body piercings, due to the length of the piercing, the curvature of the body piercing pin and the location of the piercing. Also, the rod and the securing ball are typically made of the same type of precious metal, so that the user must have an expensive separate rod and matching securing ball for each body-piercing pin, thus raising costs.

A body piercing is created by a medical piercing needle that cuts the skin away with its razor sharp edge, filling the tube of the piercing pin with the body tissue that has been cut away where the initial sterile adornment will be inserted upon extraction of the piercing pin. With all new piercings it is very important that the piercing channel be given time to heal, to grow an internal tissue or skin. Usually this healing time period varies from three months to as many as six months, depending upon the type and location of the piercing. The person that has a new piercing must keep the initial adornment in the body channel during the healing time. If the adornment is changed or removed, the piercing will take longer to heal properly.

The initial body adornment that is used in a new piercing is usually more functional than decorative. This is due to the fact that the primary purpose a new piercing adornment performs is to keep the new body piercing open during the healing process, rather than being particularly decorative, which reduces the cost for the person that installs the piercing. So, as the initial purpose of a piercing is to display a body decoration, it is not uncommon that during this initial healing period a person with a new piercing might wish to change the decorative element.

In German registered design DE29622288U1, disclosure is made of a body decoration with an exchangeable decorative element or elements, in the shape of discs having a center hole, by attaching a separate, threaded pin through to a base. One of a number of disadvantages of this system is that a special positioning pin is used, which involves additional cost and makes installation and removal complicated, as it is difficult to turn the pin in order to remove it from the threaded base. Further, the disc is usually roughly 1 mm (0.04 in.) in thickness and is uncomfortable because its edges can cut into the surrounding skin.

In U.S. Pat. No. 6,105,392 to Biagi, disclosure is made of a means of replacing an adornment that requires the decorative section to be removed, leaving the back element in place in the piercing, but able to fall out, as the back section that remains has no means of holding it in place. The back is left in the piercing to provide a guide for the replacement of the decorative element. This device, called an internal threaded labret, is common in the art, and is mainly used for piercings of the lip, where the common ball-shaped attachment might prove uncomfortable or inconvenient.

In U.S. Pat. No. 4,783,974 to Hernandez, disclosure is made of a safety device with a means to allow a “female” decorative portion of an earring to be detached, with as little as one pound of force, from a “male” wear post which remains in the ear piercing.

In U.S. Pat. No. 5,505,061 to Fleury, disclosure is made of a detachable jewelry apparatus wherein an interchanging or securing apparatus is substantially concealed so as to appear as an integral unit to a viewer.

In U.S. Pat. No. 5,438,850 to Keating, disclosure is made of a means for adapting clothing buttons to be used as jewelry, specifically, earrings. The Keating '850 patent concedes that the attachment method is not novel.

In U.S. Pat. No. 5,048,310 to Riley, disclosure is made of changeable earring pendants.

None of the above mentioned references disclose or claim a body piercing pin assembly comprising an attachment section, a body piercing section and a head section, the head section being adapted to receive a plurality of decorative sections having mounts attached. None of the references refers to the pain, discomfort and infection risk that can occur with body piercings that are larger than an earring, as none of the references are intended to be used with larger piercings. None of the references disclose a method of securely replacing decorative sections without running the risk of loss or damage to the attachment or decorative sections.

### SUMMARY

The body decoration system in accordance with the current invention includes a body-piercing section that is inserted into a body opening such that an end of the body-piercing section protrudes on either side of the body opening. The body decoration system further includes a fastening section that has a larger diameter than the body piercing section so that when these two parts are joined to each other it is not possible for the body piercing section to be pulled out or slip out in one direction. The body decoration system further includes a head section, attached to the body piercing section, for fastening a decorative section in place. The decorative section can be fastened securely to the head section, but can also be removed so that it can be exchanged with another decorative section, without needing to remove the body piercing section from the opening and without needing to detach the body piercing section from either the head section or the attachment section.

### DRAWING FIGURES

FIG. 1 illustrates an exploded illustration in diagram form of a first embodiment of the current invention, showing an exploded view of a typical decorative section and a head section which mates with the decorative section.

FIG. 2 illustrates a front view in diagram form of the first embodiment of the current invention.

FIG. 3 illustrates a side view in diagram form of the first embodiment of the current invention.

FIG. 4 illustrates an exploded illustration in diagram form of a second embodiment of the current invention.

FIG. 5 illustrates an exploded view in diagram form of a third embodiment of the current invention.

FIG. 6 illustrates a side view in diagram form of a fourth embodiment of the current invention.

FIG. 7 illustrates a side view in diagram form of a fifth embodiment of the current invention with a decorative section removed from a head section.

FIG. 8 illustrates a side view in diagram form of a fifth embodiment of the current invention with the decorative section attached to the head section.

FIG. 9 illustrates a side view in diagram form of a sixth embodiment of the current invention, wherein a body piercing section is straight and a head section is adapted to receive the straight body piercing section along the axis of symmetry of the decorative element.

FIG. 10 illustrates a side view in diagram form of a alternate sixth embodiment of the current invention, wherein a head section is provided as a separate unit that has two secondary attachments for one of two types of body piercing sections, for example: curved or straight, long or short, thin or thick. In this embodiment, a decorative section is provided which mates with the head section, allowing the user to use their own body piercing section and attachment section.

### OBJECTS AND ADVANTAGES

Accordingly, several objects and advantages of the current invention are:

(a) to create a body decorating system for application of decorative elements, by utilizing body piercings, that facilitates replacement of decorative sections, and does so in a simpler manner than is in present use;

(b) to create a body decorating system for application of decorative elements, by utilizing body piercings, that is more an economical than is in present use;

(c) to create a body decorating system for application of decorative elements, by utilizing body piercings, that reduces pain and prevents inflammation which are present in devices that are in present use;

(d) to create a body decoration system for application of decorative elements, in particular a body piercing pin assembly, that avoids or at least reduces the disadvantages of systems for body adornment that are in present use;

(e) to create a body decorating system for application of decorative elements, by utilizing body piercings, that provides a flexible system of body decoration wherein users are able to utilize existing body piercing decorations by adding a removable decorative element;

(f) to create a body decorating system for application of decorative elements, by utilizing body piercings, that allows piercings that utilize curved or straight body piercing sections to utilize removable decorative sections.

As a useful aspect of the invention, the decorative section includes a primary attachment, preferably a threaded pin or threaded cylinder, a cylindrical pin, a pin with a section of gradually increasing diameter or a bayonet-type attachment; and the head section includes a complementary primary attachment so that the head section fits together and engages the primary attachment of the decorative section. The primary attachment of the head section is preferably an internal thread, a rubber sleeve, spring element or bayonet-type attachment. In this manner the decorative section is attached to the head section, remaining attached because of the shape involved, locking action, or friction between the head section and the decorative section.

In the following portion of this application, the invention is explained in greater detail by way of sample designs, with reference made to the accompanying illustrations. The same reference symbols designate identical or similar components in these illustrations. Numerals having the same suffix amongst the different figures are intended to indicate components with substantially similar roles in other embodiments (i.e. numerals 1, 101, 201, 301, 401, and 501 are decorative sections of the first, second, third, forth, fifth and sixth embodiments respectively).

### REFERENCE NUMERALS IN DRAWINGS

| | |
|---|---|
| 1 | Decorative Section of First Embodiment |
| 2 | Decorative Element of First Embodiment |
| 3 | Decorative Element Mounting of First Embodiment |
| 4 | Ring-Shaped Fastening Section |
| 5 | Hollow Area of Mounting 3 |
| 6 | Ring-Shaped Mount Bordering Edge |
| 7 | Threaded Pin |
| 8 | Hollow Area of Head Section 9 |
| 9 | Head Section of First Embodiment |
| 10 | Primary internal Threading of Head Section 9 |
| 12 | Flattened Area of Head Section 9 |
| 13 | Diameter of Hollow Head Area 8 |
| 14 | Diameter of Bordering Ring 15 |
| 15 | Bordering Ring of Flattened Area 12 |
| 16 | Ring-Shaped Stop |
| 17 | Ring-Shaped Surface of Fastening Ring 4 |
| 18 | Internal Threading in Head for 19 |
| 19 | Body Piercing Section |
| 20 | External Threading of 19 Attaching to 18 |
| 21 | External Threading for Fastening Section 24 |
| 24 | Fastening Section |
| 25 | Primary Attachment of Fastening Section 24 |
| 26 | Body Decoration System, First Embodiment |
| 101 | Decorative Section of Second Embodiment |
| 102 | Decorative Element of Second Embodiment |
| 103 | Conical Decorative Element Mount |
| 103a | Tip of Mount 103 |
| 104 | Ring Shaped Fastening Section of 103 |
| 107 | Cylinder-Shaped Pin |
| 108 | Conical Hollow Mounting Area of 109 |
| 108a | Cylindrical Hollow Fastening Area of 109 |
| 109 | Head Section of Second Embodiment 126 |
| 110 | Resilient Sleeve of 108a |
| 111 | Cylindrical Hollow in Sleeve 110 |
| 116 | Circular Stop in Hollow Area 108 |
| 117 | Ring Shaped Surface of 104 |
| 126 | Body Decoration System, Second Embodiment |
| 201 | Decorative Section of Third Embodiment |
| 202 | Decorative Element of Third Embodiment |
| 203 | Decorative Element Mount of Third Embodiment |
| 203a | Tip of Mount 203 |
| 204 | Ring Shaped Fastening Section of 203 |
| 207 | Short Pin attached at Tip of Mount 203a |
| 207a | Ball-Shaped Fastening Section |
| 208 | Conical Hollow Area of Head Section 209 |
| 208a | Diameter of Hollow Area 208 |
| 209 | Head Section of Third Embodiment |
| 210 | Cylindrically-Shaped Hollow of Head Section 209 |
| 211 | Spring |
| 212 | Diameter of Spring 211 |
| 217 | Ring Shaped Surface of 204 |
| 226 | Body Decoration System, Third Embodiment |
| 301 | Decorative Section of Fourth Embodiment |
| 302 | Decorative Element of Fourth Embodiment |
| 303 | Threaded Decorative Element 302 Mount |
| 304 | Ring Shaped Fastening Section of 302 |
| 307 | Internal Threading of Hollow Area 308 |
| 308 | Cylindrical Hollow in 309 |
| 309 | Head Section of Fourth Embodiment |
| 316 | Circular Stop of Head Section 309 |
| 317 | Ring Shaped Surface of 304 |
| 326 | Body Decoration System, Fourth Embodiment |
| 401 | Decorative Section of Fifth Embodiment |
| 402 | Decorative Element of Fifth Embodiment |
| 403 | Cylindrical Decorative Element Mount |
| 404 | Ring Shaped Fastening Section of 403 |
| 407 | Mounting Pin of Decorative Element Mount 403 |
| 408 | Cylindrical Hollow Mounting Area of Head 409 |
| 408b | Base of Cylindrical Mounting Area 408 |
| 409 | Head Section, Fifth Embodiment |
| 410 | Cushioning Section of Mounting Area 408 |
| 411 | Attachment Groove of 408 for Pin 407a |
| 426 | Body Decoration System, Fifth Embodiment |
| 501 | Decorative Section of Sixth Embodiment |
| 509 | Head Section of Sixth Embodiment |
| 509a | Alternate Head Section of Sixth Embodiment |
| 510 | Primary Attachment of Head Section 509 |
| 518 | Secondary Attachment of Head Section 509 |
| 518a | First Alternate Secondary Attachment of 509a |
| 518b | Second Alternate Secondary Attachment of 509 |
| 519 | Body Piercing Section of Sixth Embodiment |
| 519a | Straight Body Piercing Section of Sixth Embodiment |
| 519b | Curved Body Piercing Section of Sixth Embodiment |
| 520 | Secondary Attachment of Body Piercing Section 519 |
| 520a | Secondary Attachment of 519a |
| 520b | Secondary Attachment of 519b |
| 521 | Primary Attachment of Body Piercing Section 519 |
| 524 | Fastening Section of Sixth Embodiment |
| 525 | Primary Attachment of Fastening Section 524 |
| 526 | Body Decoration System of Sixth Embodiment |

### DESCRIPTION

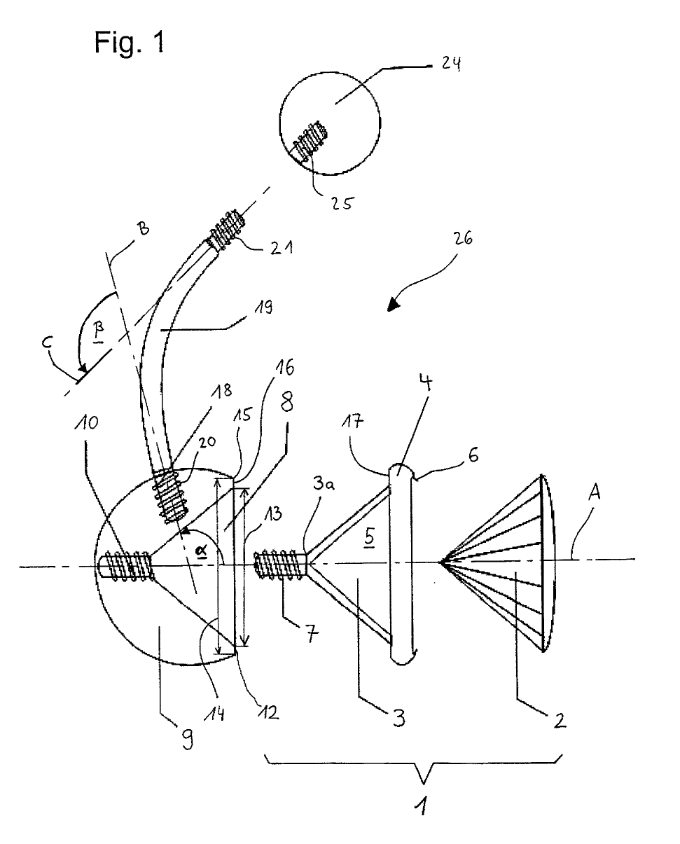

### FIGS. 1-3, FIRST EMBODIMENT: THREADEDLY ENGAGING PIN PRIMARY ATTACHMENT

FIG. 1 shows a first embodiment of a body decoration system 26, with a decorative section 1 consisting of a jewel or similar decorative element 2, for example a polished diamond, and a conical-joint-shaped mounting 3 for decorative element 2. Conical-joint-shaped mounting 3 has a ring-shaped fastening section 4 having a ring shaped edge 6 with which decorative element 2 is permanently fastened in a conical-joint-shaped hollow area 5 of conical-joint-shaped mounting 3 in a manner that is known by specialists in the area, for example by bordering edge 6 of ring-shaped fastening section 4. Conical-joint-shaped mounting 3 has a tip 3a where a primary externally threaded pin 7 is attached that runs parallel to an axis of rotational symmetry A of conical-joint-shaped mounting 3. Conical-joint-shaped mounting 3 fits in an internal hollow area 8 of a head section 9, when threaded pin 7 is screwed into place in a primary internal threading 10 in head section 9 that mates with primary external threaded pin 7.

Head section 9 has an internal cone-shaped hollow area 8 and a flattened area 12. Axis of symmetry A of internal cone-shaped hollow area 8 runs perpendicular to the level of flattened area 12. Hollow area 8 has a diameter 13 in the level of flattened area 12. Head section 9 also has a ring 15 that borders flattened area 12, having a diameter 14. Diameter 13 of hollow area 8 is somewhat smaller than diameter 14 of ring 15 that borders flattened area 12 such that a circular ring-shaped stop 16 is present for conical-joint-shaped mounting 3 in the level of flattened area 12 of head section 9. A ring-shaped surface 17 of ring-shaped fastening section 4 facing threaded pin 7 serves as a counter stop for circular ring-shaped stop 16 of head section 9.

Head section 9 has a secondary internal threading 18 that forms an angle α of about 103 degrees to axis of symmetry A of hollow area 8 and primary internal threading 10 of head section 9.

A curved body piercing section 19, having a circular cross-section, has a secondary external threading 20 that is screwed into secondary internal threading 18 of head section 9. Body piercing section 19 has a primary external threading 21. An axis B of secondary external threading 20 of body piercing section 19 and an axis C of primary external threading 21 of body piercing section 19 form an angle β of about 120 degrees as a result of the curvature of body piercing section 19.

A fastening section 24, with a primary internal threading 25, is attached to primary external threading 21 of body piercing section 19.

Conical-joint-shaped mounting 3, head section 9, body piercing section 19 and fastening section 24 consist of a precious metal, for example silver, gold, platinum or niobium or are at least coated with a precious metal. External threading 7, 20 & 21 and internal threading 10, 18 & 25 are manufactured in a similar manner.

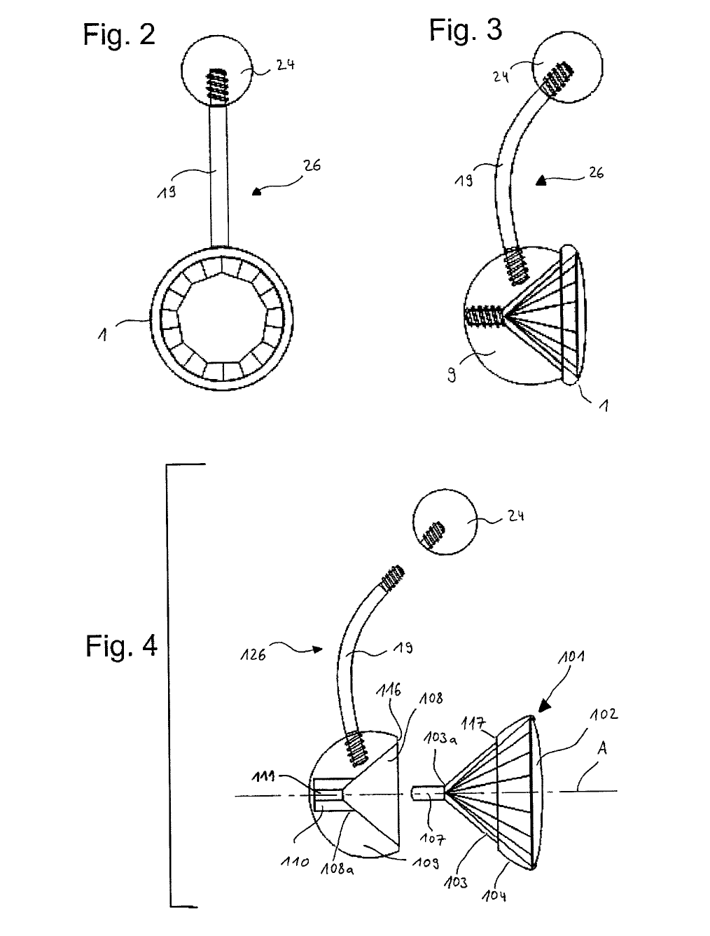

FIG. 2 shows a front view of assembled body decoration system 26 of FIG. 1 in diagram form.

FIG. 3 shows a partially transparent side view in diagram form of assembled body decoration system 26 of FIG. 1.

### FIG. 4—SECOND EMBODIMENT: FRICTIONALY ENGAGING PRIMARY ATTACHMENT

FIG. 4 shows a second embodiment of a body-piercing pin assembly 126. Body piercing section 19 and fastening section 24 are designed as they were for body decoration system 26. A decorative section 101 is comprised of a decorative element 102 and a conical mounting 103 with a tip 103a of conical mounting 103 having a cylinder-shaped pin 107 having a diameter and no thread, attached to tip 103a and centered about axis of rotational symmetry A. A ring-shaped surface 117 of a ring-shaped fastening section 104 of conical mounting 103, facing cylinder-shaped pin 107, serves as a counter stop for circular ring-shaped stop 116 of a head section 109. Decorative section 101 utilizes a standard mounting technique, as is known in the art, to attach decorative element 102 to mount 103 using ring-shaped fastening section 104.

Head section 109 forms a conical shaped hollow area 108 and a cylindrical shaped hollow fastening area 108a centered about axis of rotational symmetry A, having a resilient sleeve 110 attached to cylindrical shaped hollow fastening area 108a. Head section 109 also has a circular stop 116 bordering hollow area 108. Resilient sleeve 110 has a cylinder-shaped hollow area 111 having a diameter that is somewhat less than the diameter of cylinder-shaped pin 107, causing cylinder-shaped pin 107 to be held in place by resilient sleeve 110 by the force of friction when decorative section 101 has been inserted all the way up to circular stop 116 in conical shaped hollow area 108 of head section 109 such that ring-shaped surface 117 contacts circular stop 116.

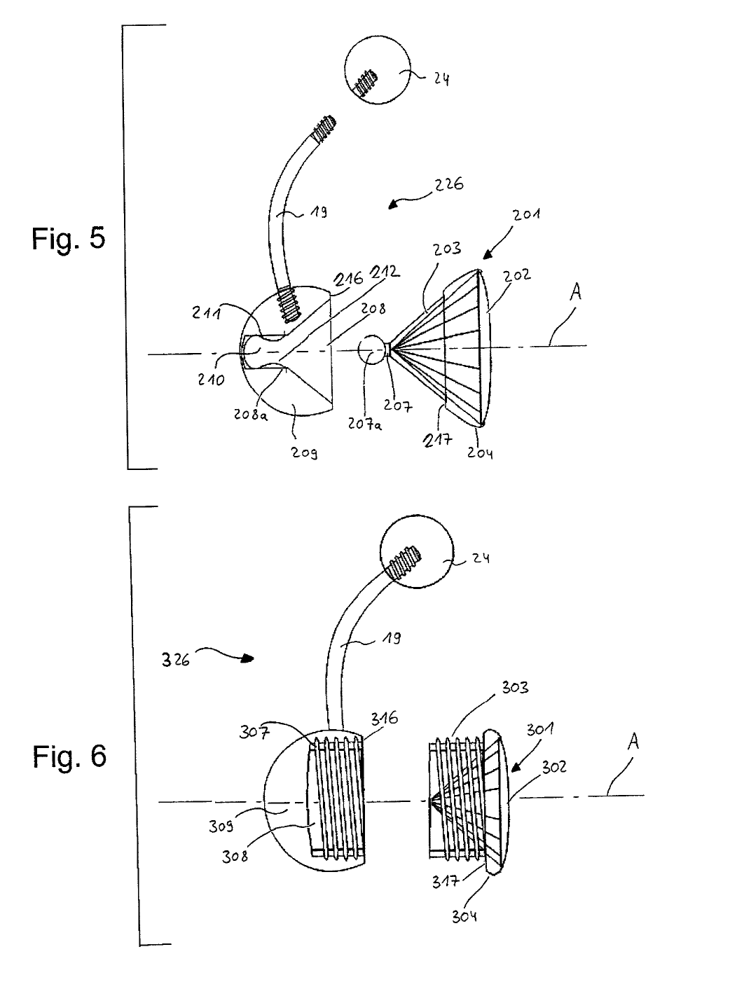

### FIG. 5—THIRD EMBODIMENT: SPRINGLY ENGAGING PIN PRIMARY ATTACHMENT

FIG. 5 shows a third embodiment of a body decoration system 226 in accordance with the current invention. Body piercing section 19 and fastening section 24 are designed as they were for body decoration system 26 & 126. A decorative section 201 is comprised of a decorative element 202 and a conical mounting 203 attached to decorative element 202. Conical mounting 203 has a tip 203a having a first end of a short, cylinder-shaped pin 207 attached to tip 203a. A ball shaped fastening section 207a with a gradually increasing diameter is centered about axis of rotational symmetry A and attached to a second end of short, cylinder-shaped pin 207. A ring-shaped surface 217 of a ring-shaped fastening section 204 of conical mounting 203 faces ball-shaped fastening section 207 and serves as a counter stop. Decorative section 201 utilizes a standard mounting technique to attach decorative element 202 to mount 203, as described in the first embodiment.

A head section 209 has a portion forming a conical shaped hollow area 208, and a portion forming a cylindrical shaped hollow fastening area 210 having a diameter 208a having a metal spring 211 having a diameter 212 that is narrower than diameter 208a about axis of rotational symmetry A such that fastening section 207a is held within cylindrical shaped hollow fastening area 210 when decorative section 201 is inserted into head section 209. Head section 209 also has a circular stop 216 bordering hollow area 208 that contacts ring shaped surface 217 of ring shaped fastening section 204 when decorative section 201 is inserted into head section 209.

### FIG. 6—FORTH EMBODIMENT: THREADEDLY ENGAGING CYLINDRICAL PRIMARY ATTACHMENT (PREFERRED EMBODIMENT)

FIG. 6 shows a forth embodiment of a body decoration system 326 in accordance with the current invention. Body piercing section 19 and fastening section 24 are designed as they were for body decoration system 26, 126, & 226. A decorative section 301 is comprised of a decorative element 302, a ring-shaped fastening section 304 attached to decorative element 302, and a cylindrical, externally threaded mounting 303 attached to ring-shaped fastening section 304 and centered about axis of rotational symmetry A. A ring-shaped surface 317 of ring-shaped fastening section 304 of decorative section 301 faces cylindrical, externally threaded mounting 303 and serves as a counter stop. Decorative section 301 utilizes a standard mounting technique to attach decorative element 302 to mount 303, as described in the first embodiment.

A head section 309 forms a cylindrical shaped hollow area 308, having an internally threaded portion 307 adapted to accept cylindrical, externally threaded mounting 303. Head section 309 also has a circular stop 316 bordering cylindrical shaped hollow area 308 that contacts ring-shaped surface 317 when decorative section 301 is threaded into head section 309. Cylindrical, externally threaded mounting 303 has a diameter that is approximately 20% less than a diameter of head section 309.

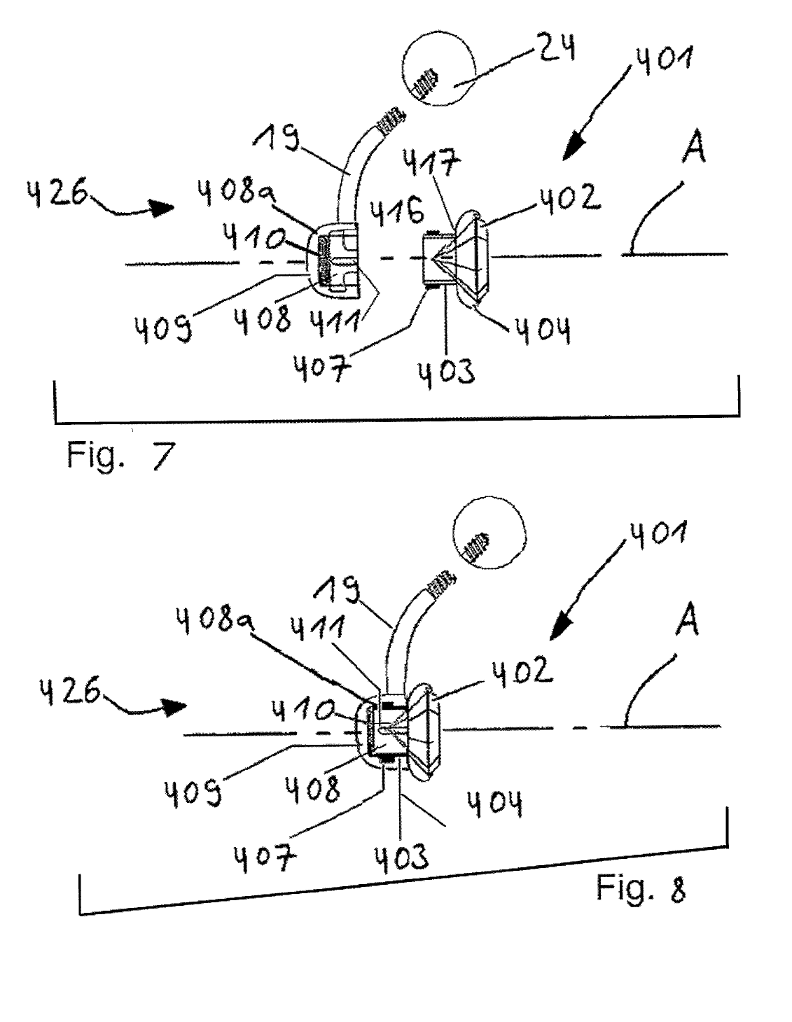

### FIGS. 7 AND 8—FIFTH EMBODIMENT: BAYONET ENGAGING PRIMARY ATTACHMENT

FIG. 7 shows a fifth embodiment of a body decoration system 426 in accordance with the current invention, in a partially unassembled form. Body piercing section 19 and fastening section 24 are designed as they were for body decoration system 26, 126, 226 & 326. A decorative section 401 is comprised of a decorative element 402, a ring-shaped fastening section 404 attached to decorative element 402, and a cylindrical mounting 403 attached to ring-shaped fastening section 404 and centered about axis of rotational symmetry A. Cylindrical mounting 403 has at least one externally attached mounting pin 407. Decorative section 401 utilizes a standard mounting technique to attach decorative element 402 to mount 403, as described in the first embodiment.

A head section 409 forms a cylindrical shaped hollow area 408, having a base 408b and at least one groove 411 which allows mounting pin 407 to be inserted into cylindrical shaped hollow area 408. Head section 409 also has a resilient cushioning section 410 attached to base 408b of head section 409. Cylindrical shaped hollow area 408 has a diameter that is approximately 20% less than a diameter of head section 409.

FIG. 8 shows the fifth embodiment of a body decoration system 426 in accordance with the current invention in assembled form. With cushioning section 410 being compressed and head section 401 being twisted such that pin 407 is captured by head section 409.

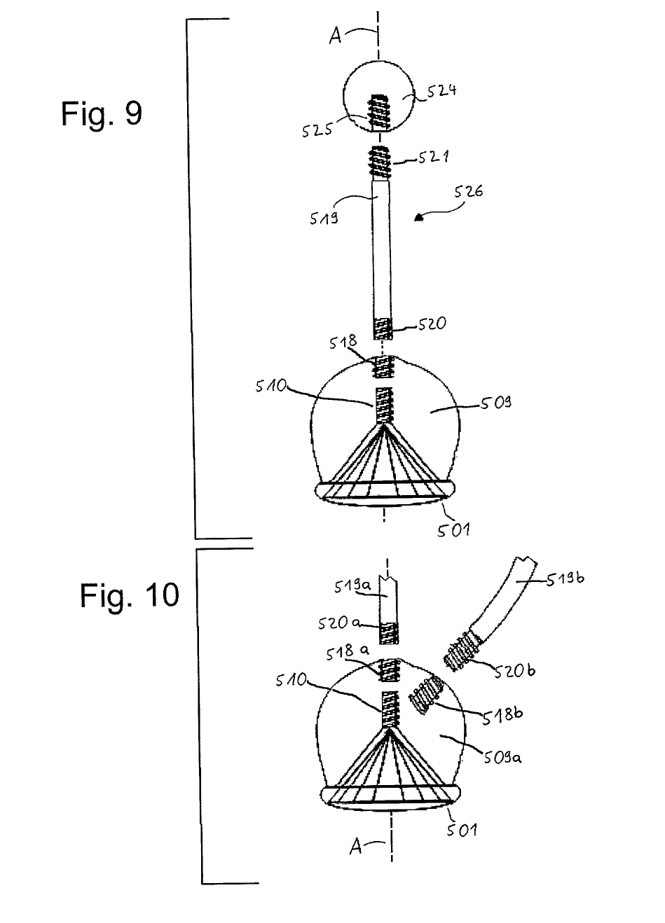

### FIG. 9—SIXTH EMBODIMENT: AXIALLY MOUNTED BODY PIERCING SECTION

FIG. 9 shows a sixth embodiment of a body decoration system 526, in accordance with the current invention. A straight body piercing section 519 has a circular cross section, a primary end 521, and a secondary end 520, forming a secondary attachment of body piercing section 519. Primary end 521 of body piercing section 519 is related in detachable mating engagement with a primary attachment 525 of a fastening section 524. Secondary external attachment 520 of body piercing section 519 is attached to a secondary internal threading 518 of a head section 509.

Head section 509 is shown with a decorative section 501 that is related in detachable mating engagement with a primary attachment 510 of head section 509 using one of the detachable mating attachment methods disclosed above. Decorative section 501, head section 509, body piercing section 519 and attachment section 525 are all substantially disposed along axis of symmetry A.

### FIG. 10 ALTERNATE SIXTH EMBODIMENT: MULTIPLE ATTACHMENT HEAD SECTION

FIG. 10 shows a seventh embodiment of a body decoration system in accordance with the current invention, wherein a head section 509a has a plurality of secondary head attachments, as exemplified by attachments 518a and 518b. Secondary plural head attachments are attached to one or more body piercing sections, exemplified by a curved body piercing section 619a and a straight body piercing section 619b.

### COMMON ELEMENTS OF INVENTIVE CONCEPT

Preferably, decorative element (i.e. 2) is essentially conical and can be fitted into an essentially conical shaped hollow area (i.e. 8) in a head section (i.e. 9) by inserting it, screwing it in, pushing it in, twisting it in, or otherwise fastening it in place.

A decorative section (i.e. 1) preferably includes a precious metal mounting and a jewel or similar decorative element (i.e. 2), for example a gem, that is permanently fastened into a mounting (i.e. 3) by some method familiar to a professional. Decorative element 2 itself may also be a precious metal adornment element.

Preferably, body piercing section 19 and 519b are designed in the shape of a curved rod with a circular cross-section, although straight rods (519, 519a) could be used with a head section (i.e. 509, 509a) wherein primary head attachment 510 and secondary head attachment 518 lie substantially along axis of symmetry A.

Preferably, body piercing section 19 and fastening section 24, for example a precious metal ball, are connected to each other but can be separated. It would be advantageous if this took place by means of an internal I external threading pair. Since especially in the case of piercing adornment, the fastening section or the securing ball also fulfills a decorative function, it will be extremely advantageous if fastening section 24 can be exchanged or removed from the body piercing section 19.

In addition, body piercing section 19 and head section 9, 109, 209, 309, 409 or 509 should preferably be permanently attached or can be connected together with an internal/external threading pair so they will also come apart.

It is especially simple in the manufacturing process to design all threads with the same specifications, which will reduce tooling costs for a manufacturer. Further, if all threads are standardized all the user would need to purchase would be a head section (i.e. 509a) that satisfied their particular need. This standardization then would lead to a further reduction of costs for the end user.

The axes of the two internal threadings (i.e. 20 and 10) in head section (i.e. 9) are at an angle greater than 90 degrees for attaching decorative section 1, 101, 201, 301, 401 or 501 and body piercing section 19, preferably between 90 degrees and 135 degrees, so that decorative section 1, 101, 201, 301, 401 or 501 is in an optimal position to the body of the user and so that is easy to put in place or to remove. This arrangement of the angle is advantageous body piercing section 19 takes the form of a curved rod, since the optimal orientation of the decorative element 2, 201, 301, or 401 is realized in this manner, that is the decorative element 2, 201, 301, 401 or 501 is facing outward so that it may be viewed. If, however, body piercing section 19 takes the form of a substantially straight rod (i.e. 519, 519a), as might be the case of an ear cartilage piercing, then the axes of the two internal threading 20 and 10 in head section 509 are at an angle of substantially 180 degrees for attaching decorative section 501 and body piercing section 519. That is to say that two internal head threadings 20 and 10 substantially lie along axis of rotational symmetry A.

In the case of a curved body piercing section 19, preferably, the tangents at either end of the body piercing section 19, and thus preferably the two axes of the external threading positioned at the ends of the body piercing section 19, are at an angle of less than 180°, preferably between 90 degrees and 180 degrees to each other. In the case of a substantially straight body piercing section 519, the tangents at either end (520, 521) of the body piercing section 519, are at an angle of substantially 180 degrees.

### OPERATION

To insert a body decoration system 26 into a body opening (not shown), fastening section 24 is detached from the body piercing section 19 and body piercing section 19 is fed through the body opening. When body piercing section 19 emerges on the other side of the body opening, fastening section 24 is reattached to body piercing section 19.

To exchange a decorative section 1, mounting 3 with decorative element 2 fastened into it is detached from head section 9 and is simply replaced by another decorative section 1.

### ADVANTAGES

The system for body decoration in accordance with the current invention makes it very easy to replace a piercing decoration, by removing a decorative section from a head section and then replacing it with another decorative section. The body piercing section remains in the body opening or the piercing hole during this time, which is advantageous. The head section and fastening section also remain attached to the body piercing section. As a result, almost any number of decorative sections can be exchanged with the basic arrangement, comprising a body piercing section, a fastening section and a head section. Different types of decorative elements can also be used, for example elements made of precious metals, with gems, enamel, or synthetic material such as colored acrylic. Different sizes of decorative elements can also be used. For example, a decorative section that utilizes a large gem could replace a decorative section that utilizes a smaller metal ball.

Pain and the risk of inflammation that can be caused by removing the body piercing sections that are in present use, are prevented in the current invention, while hygienic conditions and comfort for the user are increased. The specified system is greatly advantageous for working with parts of the body that are relatively inaccessible, for example the genitalia.

From the above description, a number of advantages of the body decoration system, according to the current invention, become apparent:

(a) replaceable decorative sections facilitate an easier method of changing a decorative section than are in present use;

(b) allowing the body piercing pin assembly to remain in the body opening while exchanging decorative sections allows for a more hygienic environment than is available with body piercing decorations that are in present use;

(c) and thus reduces the infection or abrasion risk to the body opening that can be experienced with body piercing decorations that are in present use;

(d) preventing loss or damage of an expensive decorative section, for example one having a diamond, during swimming or sports, by detaching and storing the detachable decorative section

(e) the cost of displaying a number of different decorative elements upon one's body is reduced, compared with body piercing decorations that that are in present use, as only one fastener, one body piercing section and one head section are needed to facilitate any number of diverse decorative sections.

### CONCLUSION, RAMIFICATIONS, AND SCOPE

Accordingly, the reader will see that the body piercing decoration system of the current invention produces a system that allows for fast and easy replacement of decorative sections, while providing these advantages in a more hygienic, safer and more cost effective manner than do the devices that are in present use. Furthermore, the body decoration system of the current invention has the additional advantages in that:

as the fastening section, body piercing section and head section can remain in place, the likelihood of loss of the entire system is reduced, as only the decorative section need be removed;

other attachment means could be made available to attach the head section to the decorative section;

the decorative section could integrally include the attachment means for the head section, thus removing the need for a decorative section mounting; this is especially true when the decorative element is composed of a material that can attach directly to an attachment, for example, a metal ball that is welded to a metal post, or if the material of the element can be formed directly into an attachment, such as a gem, or glass crystal that may have a post formed at the base of the gem or crystal. Further, as all threaded portions cab be similarly threaded, it would be possible to put detachable head sections on both sides of the body piercing section, thus providing attachment for two detachable decorative sections.

While specific apparatus and method have been disclosed in the proceeding description, it should be understood that these specifics have been given for the purpose of disclosing the principals of the present invention. These specifics should not be construed as limiting the scope or spirit of the invention, but merely providing illustrations of some of the presently preferred embodiments of the current invention.

It should further be understood that many variations of the current inventive concept may become apparent to those who are versed in the art of the current invention. For example, the decorative section could be manufactured as a square, cubic, spherical, or random shape, rather than as cone-shaped; the body piercing section could be large and long, such as when piercing an arm or leg, or it could be short and thin, as when used for an earring; etc.

Thus the scope of the present invention is to be determined by the appended claims and their legal equivalents, rather than by the examples given.

I claim:

1 A body decoration system for the application of decorative elements which comprises:
a body piercing section, said body piercing section having
a primary end,
a secondary end, and
a diameter;
a fastening section, said fastening section having
a diameter,
said diameter of said fastening section being greater than said diameter of said body piercing section,
a primary attachment,
said primary attachment of said fastening section being detachably related in mating engagement with said primary end of said body piercing section;
a head section, said head section having
a secondary attachment,
said secondary attachment of said head section being attached to said secondary end of said body piercing section,
a primary attachment,
said primary attachment of said head section providing a detachable mating engagement; and
a decorative section, said decorative section having
a primary attachment,
said primary attachment of said decorative section being detachably related in mating engagement to said primary attachment of said head section,
a decorative element,
said decorative element being affixed to said primary attachment of said decorative section;
whereby said decorative section may be removed and replaced with a different decorative section without removing said head section or said fastening section from said body piercing section.

2 The body decoration system according to claim 1 wherein
said decorative section includes an axis of rotational symmetry,
said primary attachment of said decorative section being disposed along said axis of said decorative section,
said head section includes an axis of rotational symmetry,
said primary attachment of said head section being disposed along said axis of said head section;
said axis of said head section lying along a central axis of rotational symmetry and
said axis of said decorative section also lying along said central axis of rotational symmetry;
said head section having a flattened area being defined by a plane which is normal to said central axis of rotational symmetry,
said flattened area having
an external diameter,
a portion forming a central hollow area,
said hollow portion having an internal diameter,
said hollow portion having a base point disposed along said axis of said head section,
said external diameter being somewhat larger than said internal diameter,
a substantially circular shaped stop area, said stop area being created between said internal diameter and said external diameter;
said primary attachment of said head section being positioned at said base point of said hollow portion in said head section;
said decorative section includes a ring-shaped fastening section attached to said decorative element;
said ring shaped fastening section being substantially centered about said central axis of rotational symmetry;
said ring shaped fasting section being in contact with said circular shaped stop area when said primary attachment of said decorative section is engaged with said primary attachment of said head section;
whereby said decorative section is strengthened and supported by said head section when said decorative section is mated with said head section.

3 The body decoration system according to claim 2 wherein
said primary attachment of said head section includes a friction element,
said friction element of said head section being frictionally related in mating engagement with said primary attachment of said decorative section.

4 The body decoration system according to claim 3 wherein
said primary attachment of said decorative section includes a pin, and
said friction element of said head section includes a material selected from the group consisting of rubber and foam rubber.

5 The body decoration system according to claim 2 wherein
said primary attachment of said decorative section includes a threaded pin, and
said primary attachment of said head section includes a primary internal threading,
said threaded pin being threadedly related in detachable mating engagement to said primary internal threading of said head section.

6 The body decoration system according to claim 2 wherein
said detachable mating engagement of said decorative section includes a section with gradually increasing diameter, and
said detachable mating engagement of said head section includes a spring element,
said section with said section with gradually increasing diameter of said detachable mating engagement of said decorative section being related in releasable locking engagement with said spring element of said detachable mating engagement of said head section.

7 The body decoration system according to claim 2 wherein
said primary attachment of said decorative section has
a first diameter,
and said decorative element of said decorative section has a second diameter,
said first diameter of said primary attachment of said decorative section and said second diameter of said decorative element of said decorative section being related by a ratio, wherein
said ratio is a ratio selected from the group consisting of 1, 1:2, 1:3, 1:5 and 1:7.

8 The body decoration system according to claim 2 wherein:
said central hollow area of said head section is essentially conical-joint-shaped,
said decorative section is essentially conical-joint-shaped,
said conically-joint-shaped decorative section including a tip, and
said primary attachment of said decorative section being attached at said tip of said conically shaped decorative section,
said tip of said conically joint shaped decorative section being detachably related in mating engagement with said primary attachment of said head section.

9 The body decoration system according to claim 2 wherein
said decorative section includes a mounting,
said mounting having
a decorative element attachment point, and
a primary attachment point,
said decorative element being permanently attached at said decorative element attachment point, and
said primary attachment of said head section being attached at said primary attachment point of said decorative section mounting.

10 The body decoration system according to claim 2 wherein
said body piercing section comprises a rod,
said rod having a circular cross-section.

11 The body decoration system according to claim 10 wherein said rod is curved.

12 The body decoration system according to claim 10 wherein said rod is straight.

13 The body decoration system according to claim 2 wherein
said primary end of said body piercing section includes an external threading, and
said primary attachment of said fastening section includes an internal threading,
said primary end of said body piercing section being removably threadedly related in mating engagement with said primary attachment of said fastening section. {add a similar claim that depends from claim 8 above}

14 The body decoration system according to claim 1 wherein
said fastening section is essentially spherical shaped.

15 The body decoration system according to claim 1 wherein
said body piercing section is a piercing pin.

16 The body decoration system according to claim 1 wherein
said secondary end of said body piercing section includes
an external threading;
said secondary attachment of said head section includes
an internal threading,;
said primary end of said body piercing section includes
an external threading;
said primary attachment of said fastening section includes
an external threading;
said external threading of said secondary end of said body piercing section being related in detachable mating engagement to said internal threading of said secondary attachment of said head section, and
said external threading of said primary end of said body piercing section being related in detachable mating engagement to said internal threading of said primary attachment of said fastening section.

17 The body decoration system according to one of claim 16 wherein
said external threading of said secondary end of said body piercing section,
said external threading of said primary end of said body piercing section,
said secondary internal threading of said head section, and
said external threading of said primary attachment of said fastening section are threaded with identical threads

18 The head section of claim 1 wherein
said primary attachment of said head section has an first axis,
said secondary attachment of said head section has a second axis,
said first axis forming an angle with said second axis,
said angle between said first axis and said second axis being preferably between 90 degrees and 135 degrees.

19 The body piercing section of claim 1, wherein
said primary end of said body piercing section has a first axis,
said secondary end of said body piercing section has a second axis,
said first axis forming an angle with said second axis, wherein
said angle is preferably less than 180 degrees,
and more preferably between 90 degrees and 180 degrees.

20 A body decoration system for the application of decorative elements which comprises:
a body piercing section, said body piercing section comprising:
a primary end having an external thread;
a secondary end having an external thread;
said body piercing section having a diameter and a circular cross section;
said body piercing section being curved;
said primary end of said body piercing section having an axis;
said secondary end of said body piercing section having an axis;
said axis of said secondary end of said body piercing section forming an angle with
said axis said primary end of said body piercing section, due to said curvature of said body piercing section;
said angle formed between said primary and secondary ends of said body piercing section is preferably less than 180 degrees, and more preferably between 90 degrees and 180 degrees;
a fastening section comprising:
a diameter,
said diameter of said fastening section being greater than said diameter of said body piercing section;
said fastening section providing a primary, threaded attachment,
said primary threaded attachment of said fastening section being threadedly related in detachable mating engagement with said primary end of said body piercing section;
a head section comprising:
an outside diameter;
a portion forming a hollow area,
said hollow area of said head section having an inside diameter,
said inside diameter of said hollow area being essentially 20% smaller than said diameter of said head section,
said hollow area of said head section having an internal thread;
said internal threading of said hollow area of said head section providing a primary attachment,
said primary attachment of said hollow area of said head section providing a detachable mating engagement;
a secondary attachment, said secondary attachment of said head section being attached to said secondary end of said body piercing section;
and a decorative section comprising:
a decorative element;
an essentially cylindrical mounting for said decorative element;
said mounting having
an outside diameter,
a top,
a bottom,
and outside surface,
a decorative element attachment being attached to said top of said mounting;
said decorative element being permanently attached to said mounting at said decorative element attachment,
said mounting having an external threading attached to said outside surface;
said diameter of said hollow area of said head section being essentially 20% less than said diameter of said decorative element section;
said external threading of said mounting providing a primary mating attachment;
said primary mating attachment of said mounting of said decorative section being removably threadedly related in mating engagement with said primary attachment of said head section.

21. A method of creating a body decoration system, comprising the steps of:
providing a body piercing section,
said body piercing section providing
a primary end,
a secondary end, and
a diameter;
providing a fastening section,
said fastening section providing a diameter,
said diameter of said fastening section being greater than said diameter of said body piercing section,
said fastening section further providing a primary attachment,
said primary attachment of said fastening section being detachably related in mating engagement with said primary end of said body piercing section;
providing a head section,
said head section providing a secondary attachment,
said secondary attachment of said head section being attached to said secondary end of said body piercing section,
said head section further providing
a primary attachment
said primary attachment of said head section providing a detachable mating engagement;
providing a decorative section,
said decorative section providing a primary mating attachment,
said primary mating attachment of said decorative section being detachably related in mating engagement to said primary attachment of said head section;
attaching said secondary attachment of said head section to said secondary attachment of said body piercing section;
attaching said primary attachment of said fastening section to said primary attachment of said body piercing section;
attaching said primary attachment of said decorative section to said primary attachment of said head section.

22 A method of attaching the body decoration system of claim 20 to a body of a wearer, comprising the steps of:
choosing said body decoration system that has a removable decorative section which exemplifies the decorative intent of said wearer;
locating a body piercing on said body,
said body piercing having,
an entry opening,
an exit opening, and
a channel connecting said entry opening and said exit opening;
detaching said primary attachment of said fastening section from said primary attachment of said body piercing section;
inserting said primary end of said body piercing section into said entry opening of said body piercing;
maneuvering said primary end of said body piercing section through said channel of said body piercing until said primary end of said body piercing section exits said channel of said body piercing via said exit opening;
reattaching said primary attachment of said body piercing section to said primary attachment of said fastening section.

23 A method of replacing the decorative section of the attached body decoration system of claim 20, comprising the steps of:
detaching said primary attachment of said decorative section from said primary attachment of said head section;
removing said decorative assembly from said head section;
selecting a new decorative assembly;
attaching said primary attachment of said new decorative assembly to said primary attachment of said head assembly.
